(19) United States
(12) Patent Application Publication
(10) Pub. No.: US 2006/0210996 A1
(43) Pub. Date: Sep. 21, 2006

(54) Methods for in situ generation of chemical arrays

(76) Inventors: Eric Leproust, San Jose, CA (US); Bill J. Peck, Mountain View, CA (US); Bruce Maeda, San Jose, CA (US)

Correspondence Address: AGILENT TECHNOLOGIES INC.;INTELLECTUAL PROPERTY ADMINISTRATION, LEGAL DEPT,, M/S DU404, P.O. BOX 7599, LOVELAND, CO, 80537-0599, US

(21) Appl. No.: 11/082,006

(22) Filed: Mar. 15, 2005

(51) Int. Cl. C12Q 1/68; G01N 1/28; G01N 33/53; B05D 3/02

(52) U.S. Cl. 435/6; 435/7.1; 427/2.11

(57) ABSTRACT

Methods and devices for producing chemical arrays are provided. Aspects of methods include employing a dedicated wash fluid reaction chamber. In certain embodiments, aspects include contacting a surface with at least a deblocking reagent to produce a deblocked surface, washing the deblocked surface in a dedicated wash fluid reaction chamber, e.g., flow cell, and then contacting the washed surface with one or more reactive moieties in a spatially controlled manner. Also provided are devices configured for use in practicing the subject methods.

### BACKGROUND OF THE INVENTION

Chemical arrays have become an increasingly important tool in the biotechnology industry and related fields. For example, nucleic acid arrays, in which a plurality of distinct or different nucleic acids are positioned on a solid support surface in the form of an array or pattern, find use in a variety of applications, including gene expression analysis, drug screening, nucleic acid sequencing, mutation analysis, and the like.

A feature of many chemical arrays that have been developed is that each of the distinct chemical moieties of the array is stably attached to a discrete location on the array surface, such that its position remains constant and known throughout the use of the array. Stable attachment is achieved in a number of different ways, including covalent bonding of the moiety to the support surface and non-covalent interaction of the moiety with the surface.

For example, with respect to nucleic acid arrays, there are two main methods of immobilizing nucleic acids by covalent bonding of the moiety to the substrate surface, i.e., via in situ synthesis in which the nucleic acid ligand is grown on the surface of the substrate in a step-wise fashion and via deposition of the full ligand, e.g., a presynthesized nucleic acid/polypeptide, cDNA fragment, etc., onto the surface of the array.

Where the in situ synthesis approach is employed, conventional phosphoramidite synthesis protocols are typically used. In phosphoramidite synthesis protocols, the 3′-hydroxyl group of an initial 5′-protected nucleoside is first covalently attached to the polymer support, e.g., a planar substrate surface. Synthesis of the nucleic acid then proceeds by deprotection of the 5′-hydroxyl group of the attached nucleoside, followed by coupling of an incoming nucleoside-3′-phosphoramidite to the deprotected 5′ hydroxyl group (5′-OH). The resulting phosphite triester is finally oxidized to a phosphotriester to complete the internucleotide bond. The steps of deprotection, coupling and oxidation may be repeated until a nucleic acid of the desired length and sequence is obtained. Optionally, a capping reaction may be used after the coupling and/or after the oxidation to inactivate the growing DNA chains that failed in the previous coupling step, thereby avoiding the synthesis of inaccurate sequences.

As chemical arrays are used more and continue to play important roles in a variety of applications, there continues to be an interest in the development of methods of manufacturing chemical arrays.

### SUMMARY OF THE INVENTION

Methods and devices for producing chemical arrays are provided. Aspects of methods include employing a dedicated wash fluid reaction chamber. In certain embodiments, aspects include contacting a surface with at least a deblocking reagent to produce a deblocked surface, washing the deblocked surface in a dedicated wash fluid reaction chamber, e.g., flow cell, and then contacting the washed surface with one or more reactive moieties in a spatially controlled manner. Also provided are devices configured for use in practicing the subject methods.

### BRIEF DESCRIPTIONS OF THE DRAWINGS

FIG. 1 shows an exemplary substrate carrying an array, such as may be used in the devices of the subject invention.

FIG. 2 shows an enlarged view of a portion of FIG. 1 showing spots or features.

FIG. 3 is an enlarged view of a portion of the substrate of FIG. 1.

FIG. 4 is a schematic diagram depicting an embodiment of an apparatus for producing a chemical array according to an embodiment of the subject invention.

FIGS. 5A and 5B provide the results of an assay that employed an array that was not fabricated using a dedicated wash flow cell and an assay that employed an array that was fabricated using a dedicated wash flow cell, as reviewed in greater detail in the experimental section below.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The term “biomolecule” means any organic or biochemical molecule, group or species of interest that may be formed in an array on a substrate surface. Exemplary biomolecules include peptides, proteins, amino acids and nucleic acids.

The term “peptide” as used herein refers to any compound produced by amide formation between a carboxyl group of one amino acid and an amino group of another group.

The term “oligopeptide” as used herein refers to peptides with fewer than about 10 to 20 residues, i.e. amino acid monomeric units.

The term “polypeptide” as used herein refers to peptides with more than about 10 to about 20 residues. The terms “polypeptide” and “protein” may be used interchangeably.

The term “protein” as used herein refers to polypeptides of specific sequence of more than about 50 residue and includes D and L forms, modified forms, etc.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “nucleoside” and “nucleotide” are intended to include those moieties that contain not only the known purine and pyrimidine base moieties, but also other heterocyclic base moieties that have been modified. Such modifications include methylated purines or pyrimidines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “ribonucleic acid” and “RNA” as used herein refer to a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

A “biopolymer” is a polymer of one or more types of repeating units. Biopolymers are typically found in biological systems (although they may be made synthetically) and may include peptides or polynucleotides, as well as such compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. For example, a “biopolymer” may include DNA (including cDNA), RNA, oligonucleotides, and PNA and other polynucleotides as described in U.S. Pat. No. 5,948,902 and references cited therein (all of which are incorporated herein by reference), regardless of the source.

A “biomonomer” references a single unit, which can be linked with the same or other biomonomers to form a biopolymer (e.g., a single amino acid or nucleotide with two linking groups, one or both of which may have removable protecting groups).

An “array,” or “chemical array” used interchangeably includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays may be fabricated using drop deposition from spatially controlled fluid deposition elements, e.g., pulse-jets, of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein.

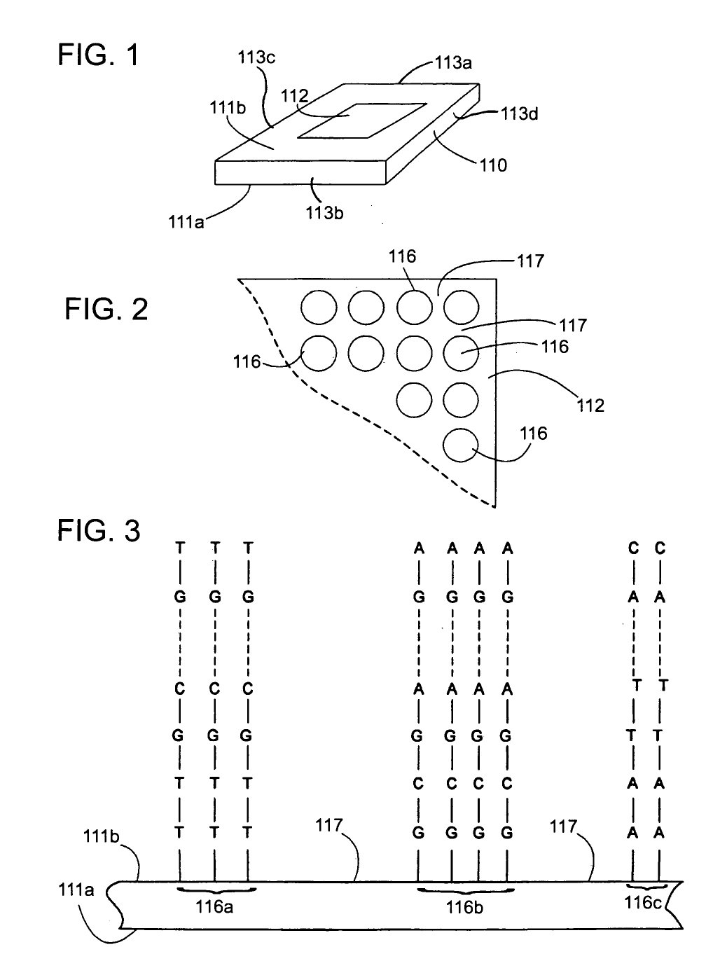

An exemplary chemical array is shown in FIGS. 1-3, where the array shown in this representative embodiment includes a contiguous planar substrate 110 carrying an array 112 disposed on a rear surface 111b of substrate 110. It will be appreciated though, that more than one array (any of which are the same or different) may be present on rear surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymers, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on front surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

An array “assembly” includes a substrate and at least one chemical array, e.g., on a surface thereof. Array assemblies may include one or more chemical arrays present on a surface of a device that includes a pedestal supporting a plurality of prongs, e.g., one or more chemical arrays present on a surface of one or more prongs of such a device. An assembly may include other features (such as a housing with a chamber from which the substrate sections can be removed). “Array unit” may be used interchangeably with “array assembly”.

The term “monomer” as used herein refers to a chemical entity that can be covalently linked to one or more other such entities to form a polymer. Of particular interest to the present application are nucleotide “monomers” that have first and second sites (e.g., 5′ and 3′ sites) suitable for binding to other like monomers by means of standard chemical reactions (e.g., nucleophilic substitution), and a diverse element which distinguishes a particular monomer from a different monomer of the same type (e.g., a nucleotide base, etc.). In the art synthesis of nucleic acids of this type utilizes an initial substrate-bound monomer that is generally used as a building-block in a multi-step synthesis procedure to form a complete nucleic acid.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other polynucleotides which are C-glycosides of a purine or pyrimidine base. In the practice of the instant invention, oligomers will generally comprise about 2-60 monomers, preferably about 10-60, more preferably about 50-60 monomers.

“Activator” refers to any suitable chemical and/or physical entity that is employed to make-possible, assist, enhance or increase in the joining or linking of a monomer to another chemical entity such as one or more other monomers or a reactive functional group such as a free hydroxy functional group present on a substrate surface, etc. For example, an activator may protonate a monomer so that it may be joined to another monomer or to a free functional group. For example, activators may be employed in phosphoramidite chemistry where they used in the joining of a deoxynucleoside phosphoramidite to a functional group present on a substrate surface or to another deoxynucleoside phosphoramidite. In producing nucleic acids on a substrate surface using phosphoramidite chemistry, one of the first steps in such a protocol involves attaching a first monomer to the substrate surface. Accordingly, a solution containing a protected deoxynucleoside phosphoramidite and an activator, such as tetrazole, benzoimidazolium triflate (“BZT”), S-ethyl tetrazole, and dicyanoimidazole, is applied to the surface of a substrate that has been chemically prepared to present reactive functional groups such as, for example, free hydroxyl groups. The activators tetrazole, BZT, S-ethyl tetrazole, and dicyanoimidazole are acids that protonate the amine nitrogen of the phosphoramidite group of the deoxynucleoside phosphoramidite. A free hydroxyl group on the surface of the substrate displaces the protonated secondary amine group of the phosphoramidite group by nucleophilic substitution and results in the protected deoxynucleoside covalently bound to the substrate via a phosphite triester group. An analogous methodology using an activator may be employed to link two deoxynucleoside phosphoramidites together such as a deoxynucleoside phosphoramidite to a substrate bound nucleotide. For example, a protected deoxynucleoside phosphoramidite in solution with an activator is applied to the substrate-bound nucleotide and reacts with the 5′ hydroxyl of the nucleotide to covalently link the protected deoxynucleoside to the 5′ end of the nucleotide via a phosphite triester group. In accordance with the subject invention, suitable “activators” include, but are not limited to, tetrazole and tetrazole derivatives such as S-ethyl tetrazole, dicyanoimidazole (“DCI”), benzimidazolium triflate (“BZT”), and the like. Activators are usually, though not always, present in a liquid, typically in solution, where such may be referred to as a “fluid activator”. In describing the subject invention, an activator includes an activator alone or with a suitable medium such as a fluid medium or the like. As such, an activator and a fluid activator may be used interchangeably herein.

The term “sample” as used herein relates to a material or mixture of materials, typically, although not necessarily, in fluid form, containing one or more components of interest.

The terms “protection” and “deprotection” as used herein relate, respectively, to the addition and removal of chemical protecting groups using conventional materials and techniques within the skill of the art and/or described in the pertinent literature; for example, reference may be had to Greene et al., Protective Groups in Organic Synthesis, 2nd Ed., New York: John Wiley & Sons, 1991. Protecting groups prevent the site to which they are attached from participating in the chemical reaction to be carried out.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

When two items are “associated” with one another they are provided in such a way that it is apparent one is related to the other such as where one references the other. For example, an array identifier can be associated with an array by being on the array assembly (such as on the substrate or a housing) that carries the array or on or in a package or kit carrying the array assembly. “Stably attached” or “stably associated with” means an item's position remains substantially constant where in certain embodiments it may mean that an item's position remains substantially constant and known.

A “web” references a long continuous piece of substrate material having a length greater than a width. For example, the web length to width ratio may be at least 5/1, 10/1, 50/1, 100/1, 200/1, or 500/1, or even at least 1000/1.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

“Rigid” refers to a material or structure which is not flexible, and is constructed such that a segment about 2.5 by 7.5 cm retains its shape and cannot be bent along any direction more than 60 degrees (and often not more than 40, 20, 10, or 5 degrees) without breaking.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

“Contacting” means to bring or put together. As such, a first item is contacted with a second item when the two items are brought or put together, e.g., by touching them to each other.

“Depositing” means to position or place an item at a location-or otherwise cause an item to be so positioned or placed at a location. Depositing includes contacting one item with another. Depositing may be manual or automatic, e.g., “depositing” an item at a location may be accomplished by automated robotic devices.

By “remote location,” it is meant a location other than the location at which the array (or referenced item) is present and hybridization occurs (in the case of hybridization reactions). For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating” information means transmitting the data representing that information as signals (e.g., electrical, optical, radio signals, and the like) over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

An array “package” may be the array plus only a substrate on which the array is deposited, although the package may include other features (such as a housing with a chamber).

A “chamber” references an enclosed volume (although a chamber may be accessible through one or more ports). It will also be appreciated that throughout the present application, that words such as “top,” “upper,” and “lower” are used in a relative sense only.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that many computer-based systems are available which are suitable for use in the present invention. The data storage means may comprise any manufacture comprising a recording of the present information as described above, or a memory access means that can access such a manufacture.

A “processor” references any hardware and/or software combination which will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

“Computer readable medium” as used herein refers to any storage or transmission medium that participates in providing instructions and/or data to a computer for execution and/or processing. Examples of storage media include floppy disks, magnetic tape, UBS, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer. A file may be stored in permanent memory.

With respect to computer readable media, “permanent memory” refers to memory that is permanently stored on a data storage medium. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “memory” or “memory unit” refers to any device which can store information for subsequent retrieval by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

Items of data are “linked” to one another in a memory when the same data input (for example, filename or directory name or search term) retrieves the linked items (in a same file or not) or an input of one or more of the linked items retrieves one or more of the others.

It will also be appreciated that throughout the present application, that words such as “cover”, “base” “front”, “back”, “top”, are used in a relative sense only. The word “above” used to describe the substrate and/or flow cell is meant with respect to the horizontal plane of the environment, e.g., the room, in which the substrate and/or flow cell is present, e.g., the ground or floor of such a room.

A “reaction chamber” according to the subject invention is an enclosed space suitable for use in the subject protocols. In certain embodiments, the reaction chamber is a flow cell. A flow cell may be described broadly as having a housing that forms a chamber where an array substrate may be positioned, as reviewed in greater detail below.

### DETAILED DESCRIPTION OF THE INVENTION

Methods and devices for producing chemical arrays are provided. Aspects of methods include employing a dedicated wash fluid reaction chamber. In certain embodiments, aspects include contacting a surface with at least a deblocking reagent, e.g., following an oxidation step) to produce a deblocked surface, washing the deblocked surface in a dedicated wash fluid reaction chamber, e.g., flow cell, and then contacting the washed surface with one or more reactive moieties in a spatially controlled manner. Also provided are devices configured for use in practicing the subject methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, aspects of the subject invention provide methods of producing chemical arrays, including nucleic acid arrays. Embodiments of the subject invention include methods of producing chemical arrays by in situ synthesis of two or more distinct chemical polymers on the surface of a solid support. The in situ synthesis protocol employed in certain embodiments of the subject invention may be viewed as an iterative process that includes two or more cycles, where each cycle includes the following steps: (1) a functional group generation step in which surface attachment moieties are generated on the surface of a substrate (i.e., solid support), e.g., by at least contacting the surface with a deblocking agent, e.g., in a non-spatially controlled manner (where the deblocking agent step may be preceded by an oxidizing step); and (2) a moiety, e.g., monomer, attachment step in which two or more different moieties, such as blocked monomers, are covalently bonded to two or more distinct locations (but not the entire surface such that they are contacted with the surface in a spatially controlled manner), e.g., at least a first and second location, of the deblocked substrate surface. In yet other embodiments, a variation of the above protocol is employed, in which the surface of the substrate is deblocked in a spatially controlled manner, e.g., via use of photolabile blocking groups and selective irradiation of the surface. These embodiments may be viewed as an iterative process that includes two or more cycles, where each cycle includes the following steps: (1) a functional group generation step in which surface attachment moieties are generated on the surface of a substrate (i.e., solid support), e.g., by at least contacting selected portions or regions of the the surface with a deblocking agent, such as light, in a spatially controlled manner (where again the deblocking agent step may be preceded by an oxidizing step); and (2) a moiety, e.g., monomer, attachment step in which two or more different moieties, such as blocked monomers, are covalently bonded to two or more distinct locations, e.g., at least a first and second location, of the selectively deblocked substrate surface, where the moities may be attached by contacting the entire surface of the substrate.

Aspects of the invention include a washing step performed between any two steps, e.g., between two steps in which a surface previously contacted with a deblocking agent is washed with a wash fluid in a dedicated wash fluid reaction chamber, e.g., a flow cell. As this reaction chamber is dedicated to contact of the surface with wash fluid, it is employed solely for contacting the substrate surface with wash fluid, as described in greater detail below.

Each of these cycle steps of the above representative embodiments is now described separately in greater detail in terms of these particular embodiments. However, the scope of the invention is not so limited, as the invention being described in terms of these particular representative embodiments is for ease of description only.

In the functional group generation step of the subject methods, surface attachment moities, i.e., reactive functional groups, are generated on a surface of the substrate, which reactive functional groups are then employed in the moiety attachment step to covalently bond moieties, e.g., reactive monomers, to the substrate surface. As the subject methods are iterative processes, functional group generation steps are typically performed following a reactive moiety deposition step, so that the synthesis cycle can be repeated with a new round of activated, blocked nucleoside monomers.

In many embodiments of the subject methods, the functional group generation step at least includes contacting a surface of a substrate (e.g., a surface on which blocked moieties have previously been deposited, such as described below) with a deblocking agent, e.g., to remove blocking groups present on the substrate surface and thereby generate reactive functional groups. In representative embodiments, the functional group generation step includes contacting the substrate surface with at least an oxidizing agent and a deblocking agent. In certain embodiments, the surface may also be contacted with a capping agent. Representative embodiments of each of these steps is described in greater detail below. As reviewed above, depending on the particular protocol the deblocking may be non-spatially or spatially controlled.

In certain embodiments, one or more of the substeps of the functional group generation step may be accomplished by contacting the entire surface of the substrate with an appropriate agent, e.g., an oxidation agent, a capping agent, a deblocking agent, etc. Contact of the entire surface may be achieved in the subject methods in a non-spatially controlled manner, e.g., by flooding the substrate surface with the appropriate agent. Conveniently, a reaction chamber (e.g., flow cell) approach may be employed, such that the entire substrate is contacted with a volume of the appropriate agent in liquid form, e.g., by flowing a volume of the appropriate liquid over the surface of the substrate in an appropriate container or chamber, e.g., such as a flow cell. In such embodiments, performance of each substep includes flowing an adequate volume of the appropriate fluid over the substrate surface so that the entire surface of the substrate is contacted with the fluid. The above step results in generation of functional groups on the surface of the substrate, where the substrate is conveniently referred to as a substrate having a deblocked surface.

Where the functional group generation step includes contacting a substrate surface with a plurality of two or more different reagents, where one of the reagents is a deblocking reagent, the two or more different reagents may be contacted with the substrate surface in the same or different reaction chamber. For example, in one embodiment, the oxidation and deblocking substeps (as well as capping substep, if employed) are all carried out in the same reaction chamber, which reaction chamber is different from the dedicated wash reaction chamber, as reviewed below. In another embodiment, the oxidation and deblocking substeps (as well as capping substep, if employed) are all performed in different dedicated reaction chambers, e.g., a dedicated oxidizing agent reaction chamber, a dedicated deblocking agent reaction chamber, a dedicated capping agent dedicated reaction chamber, etc., such that the reaction chamber used to perform one substep (e.g., contacting the substrate surface with a oxidizing agent) is different that the reaction chamber used to perform a subsequent substep (e.g., contacting the substrate surface with a deblocking agent). In yet another embodiment, any combination of the substeps may be performed in a single reaction chamber, so long as the washing substep is performed in a dedicated reaction chamber, i.e., a reaction chamber that is not used to contact the substrate surface with another agent, such as an oxidizing agent, capping agent, deblocking agent, etc. For example, the oxidation and optional capping substeps may be performed in a first reaction chamber, the deblocking substep may be performed in a second reaction chamber, and the washing substep is performed in a third reaction chamber, which is the dedicated wash fluid reaction chamber.

In some embodiments, where a first reaction chamber is used to contact the substrate surface with more than one reagent, the functional group generation step includes a process in which the substrate surface is sequentially contacted or flooded with a plurality of two or more different fluids, for example three or more fluids, including four or more fluids, such as oxidizing fluid, wash fluid, deblocking fluid, and optionally capping fluid, as reviewed in greater detail below. In another embodiment, each substep (e.g., the oxidation, optional capping, and deblocking substeps) is performed in different reaction chambers, i.e., the reaction chamber used to perform one substep (e.g., contacting the substrate surface with an oxidizing agent) is different that the reaction chamber used to perform a subsequent substep (e.g., contacting the substrate surface with a deblocking agent). Accordingly, as described above certain embodiments may include sequential contact of a substrate surface with the following liquid agents in the following order in a first reaction chamber: (1) oxidizing agent; (2) optional washing agent; and (3) deblocking agent, (4) optional washing agent, where the substrate is contacted with each of these agents in the same reaction chamber or different reaction chambers.

In the subject methods, the resultant substrate having the deblocked surface is then washed to produce a substrate having a washed deblocked surface, where the washed deblocked surface displays the desired functional groups employed in the moiety attachment step. Washing of the deblocked surface is conveniently performed by transporting the substrate to wash fluid dedicated reaction chamber (e.g., flow cell), where the substrate surface is contacted with a wash fluid. As the wash fluid reaction chamber employed in this step is a dedicated wash fluid reaction chamber, it is employed solely for contact of wash fluid with a substrate surface. As such, it is not used to contact a substrate surface with another agent, such as an oxidizing agent, capping agent, deblocking agent, etc. A feature of the dedicated wash fluid reaction chamber is that it is operatively coupled to a source of wash fluid, but not to a source of any other type of reagent. By operatively coupled is meant that it is in fluid communication (which may be turned on or off) with a reservoir of wash fluid.

Following the above steps, the substrate may be transported from the second reaction chamber (e.g., a flow cell) to a fluid deposition station (e.g., a reactive moiety addition station) such as a printing chamber or the like, where moiety addition to the surface is carried out. In representative embodiments, the fluid deposition station is a spatially controlled fluid deposition station. By spatially controlled fluid deposition station is meant that the deposition station deposits fluid on the substrate surface to predetermined and defined locations of the surface. Representative spatially controlled fluid deposition stations are described in greater detail below.

The above steps of: (a) functional group generation; (b) washing; and (c) moiety attachment; may be repeated a number of times with additional reactive moieties, e.g., nucleotides, until each of the desired chemical moieties, e.g., polymeric ligands, such as nucleic acids or polypeptides, is produced on the substrate surface. For example, by choosing which sites are contacted with which activated nucleotides, e.g. A, G, C & T, an array having nucleic acid polymers of desired sequence and spatial location is readily achieved. As such, the above cycles of reactive moiety attachment and functional (e.g., hydroxyl) moiety regeneration result in the production of an array of desired chemical moieties, e.g., polymers, such as nucleic acids. The resultant chemical arrays can be employed in a variety of different applications, as described in greater detail below.

As reviewed above, alternative embodiments of the above may vary from the above in one or more ways. For example, in certain alternative embodiments, the blocking groups may be photo-cleavable, such that following oxidation, regions of the surface are selectively irradiated to deblock the surface in a spatially controlled manner. Photocleavable blocking groups and irradiation protocols employed to cleave the same are described in, among other publications, published United States Application 20040265476. These embodiments provide for the ability to contact the entire surface of the substrate with the reactive moieties, e.g., in a non-spatially controlled manner.

The above method steps may be carried out manually or with a suitable automated device, where in many embodiments a suitable automated device is employed. Of particular interest is an automated device that is adapted to automatically transfer a substrate from an fluid deposition element for depositing a reactive moiety on a substrate surface in a spatially controlled manner, i.e., a “writer station”, to a surface processing station that includes at least the following two substations: (a) functional group generation station where functional group generation, e.g., via contacting a surface with at least a deblocking agent, is carried out in a least a reagent contact reaction chamber; and (b) a washing station where washing is carried out in a dedicated wash station. In these automated embodiments, when moving a substrate between the fluid deposition element and the fluid reaction chambers, the substrate may be transported by a transfer element such as a robotic arm, and so forth. In one embodiment, a transfer robot is mounted on a platform of an apparatus used in the synthesis. The transfer robot may include a base, an arm that is movably mounted on the base, and a grasping element adapted to grasp the substrate during transport that is attached to the arm. The element for grasping the substrate may be, for example, movable finger-like projections, and the like. In one aspect, in use, the robotic arm is activated so that the substrate is grasped by the grasping element. The arm of the robot is moved so that the substrate is delivered to the flow cell.

In the subject methods and devices, any convenient reaction chamber may be employed. A reaction chamber suitable for use with the subject invention is a flow cell. A flow cell may be described broadly as having a housing that forms a chamber where an array substrate may be positioned. As summarized above, the flow cell allows fluids to be passed through the flow cell chamber where the array substrate is disposed. The array substrate is mounted in the chamber in or on a holder. The flow cell housing usually further includes at least one fluid inlet and at least one fluid outlet for flowing fluids into and through the chamber in which the support is mounted. In one approach, the fluid outlet may be used to vent the interior of the reaction chamber for introduction and removal of fluid by means of the inlet. On the other hand, fluids may be introduced into the reaction chamber by means of the inlet with the outlet serving as a vent and fluids may be removed from the reaction chamber by means of the outlet with the inlet serving as a vent.

The dimensions of the housing chamber of the employed flow cell may vary and are dependent on the dimensions of the support that is to be placed therein. In certain embodiments, the array substrate may be one on which a single array of chemical compounds is synthesized. In this regard the substrate may range from about 1.5 to about 5 inches in length and about 0.5 to about 3 inches in width. The substrate may range from about 0.1 to about 5 mm, e.g., about 0.5 to about 2 mm, in thickness. A standard size microscope slide is usually about 3 inches in length and 1 inch in width and may be used. Alternatively, multiple arrays of chemical compounds may be synthesized on a given substrate or wafer, which may be used as is or which may then be diced, i.e., cut, into single array substrates in which each dices section may include one or more chemical arrays. In this alternative approach the substrate may range from about 5 to about 8 inches in length and about 5 to about 8 inches in width so that the substrate may be diced into multiple single array substrates having the aforementioned dimensions. The thickness of the substrate may be the same as that described above. In a specific embodiment by way of illustration and not limitation, a substrate that has dimensions of about 6⅝ inches by about 6 inches may be employed and diced into about 1 inch by about 3 inch substrates.

Representative flow cells that may be employed in certain embodiments may be about 6.5 inches wide by about 6 inches tall in the plane of the flow cell. More generally these dimensions may range from the size of an array about 1 cm square to about 1 meter square. The gap width in representative embodiments of flow cells that may be employed in the invention may range from about 1 μm to about 500 μm, and in certain embodiments may range from about 1-10 μm to about 10 mm.

Flow cell devices employed in array fabrication which may be adapted for use with the subject invention are further described in, for example, U.S. Published Patent Application Nos. 20040180450; 20030003222; 20030003504; 20030112022; 200030228422; 200030232123; and 20030232140; and U.S. Pat. No. 6,713,023.

The housing of the flow cell is generally constructed to permit access into the chamber therein. The flow cell may have an opening that is sealable to fluid transfer after the array substrate is placed therein. Such seals may include a flexible material that is sufficiently flexible or compressible to form a fluid tight seal that may be maintained under increased pressures encountered in the use of the device. The flexible member may be, for example, rubber, flexible plastic, flexible resins, and the like and combinations thereof. In any event the flexible material should be substantially inert with respect to the fluids introduced into the device and must not interfere with the reactions that occur within the device. The flexible member may be a gasket and may be in any shape such as, for example, circular, oval, rectangular, and the like, e.g., the flexible member may be in the form of an O-ring in certain embodiments.

Alternatively, the housing of the flow cell may be conveniently constructed in two parts, which may be referred to generally as top and bottom elements. These two elements are sealably engaged during synthetic steps and are separable at other times to permit the support to be placed into and removed from the chamber of the flow cell. Generally, the top element is adapted to be moved with respect to the bottom element although other situations are contemplated herein. Movement of the top element with respect to the bottom element may be achieved by means of, for example, pistons, and so forth. The movement may be controlled electronically by means that are conventional in the art. In another approach a reagent chamber may be formed in situ from an array substrate and a sealing member. The inlet of the flow cell is usually in fluid communication with an element that controls the flow of fluid into the flow cell such as, for example, a manifold, a valve, and the like or combinations thereof. This element, in turn, is in fluid communication with one or more fluid reagent dispensing stations. In this way different fluid reagents for one step in the synthesis of the chemical compound may be introduced sequentially into the flow cell.

In one embodiment, the fluid dispensing stations may be affixed to a base plate or main platform to which the flow cells are mounted. Any fluid dispensing station may be employed that dispenses fluids such as water, aqueous media, organic solvents, ionic liquids and the like. The fluid dispensing station may include a pump for moving fluid and may also comprise a valve assembly and a manifold as well as a means for delivering predetermined quantities of fluid to the flow cell. The fluids may be dispensed by pumping from the dispensing station. In this regard, any standard pumping technique for pumping fluids may be employed in the present apparatus. For example, pumping may be by means of a peristaltic pump, a pressurized fluid bed, a positive displacement pump, e.g., a syringe pump, and the like.

After a reagent is introduced into the flow cell, the reagent is held in contact with the array substrate for a time and under conditions sufficient for the particular step to be completed. The time periods and conditions are dependent on the nature of the reagent and the nature of the particular step of the procedure. For example, the time periods and conditions may be different for a washing procedure rather than an oxidizing reaction or a deblocking reaction. In general, the time periods and conditions for the procedures conducted in the flow cells are well-known in the art and will not be repeated here.

The amount of the reagents employed in each of the above steps in the method of the present invention is dependent on the nature of the reagents, solubility of the reagents, reactivity of the reagents, availability of the reagents, purity of the reagents, and so forth. Such amounts should be readily apparent to those skilled in the art in view of the disclosure herein. In one aspect, stoichiometric amounts are employed; however, in other aspects excess of one reagent over the other may be used where circumstances dictate. Typically, the amounts of the reagents are those necessary to achieve the overall synthesis of the chemical compound, which may be, e.g., a nucleic acid as described herein, in accordance with the present invention. The time period for conducting the present method is dependent upon the specific reaction and reagents being utilized and the chemical compound being synthesized.

In further describing the subject invention, a representative embodiment in which the methods are employed to fabricate a nucleic acid array is now reviewed in greater detail. It should be noted that the following nucleic acid array fabrication description is merely exemplary. Various modifications to the following description may be made and still fall within the scope of the invention. For example, the “direction” of synthesis may be reversed, such that the synthesized nucleic acids are attached to the substrate at their 5′ ends and one generates 3′ functional groups in the deblocking/deprotecting step and certain fluids may be omitted and/or certain fluids may be added to the sequence.

In these representative embodiments, the synthesis of arrays of polynucleotides on the surface of a support includes two steps: (a) 5′-OH functional group generation step; (b) a washing step; and (c) a blocked monomer attachment step. The protocol in these representative embodiments may be viewed as iterative, in the following steps are repeated two or more times to produce a nucleic acid array: (i) coupling an activated selected nucleoside (a monomeric unit) through a phosphite linkage to a functionalized support in the first iteration, or a nucleoside bound to the substrate (i.e. the nucleoside-modified substrate) in subsequent iterations; (ii) optionally, blocking unreacted hydroxyl groups on the substrate bound nucleoside (sometimes referenced as “capping”); (iii) oxidizing the phosphite linkage of step (i) to form a phosphate linkage; (iv) removing the protecting group (“deprotection”) from the now substrate bound nucleoside coupled in step (i), to generate a reactive site for the next cycle of these steps; and (v) washing the resultant deprotected (i.e., deblocked surface) with a wash fluid in a dedicated wash fluid reaction chamber. The coupling can be performed by depositing drops of an activator and phosphoramidite at the specific desired feature locations for the array. Capping, oxidation and deprotection can be accomplished by treating the entire substrate with a layer of the appropriate reagent such as sequentially flowing the particular reagent(s) across the substrate surface, for example in a flow cell system. The functionalized support (in the first cycle) or deprotected coupled nucleoside (in subsequent cycles) provides a substrate bound moiety with a linking group for forming the phosphite linkage with a next nucleoside to be coupled in step (i). Final deprotection of nucleoside bases can be accomplished using alkaline conditions such as ammonium hydroxide, in another reagent contacting step such as described above in a flow cell system.

To produce nucleic acid arrays according to this representative embodiment, a substrate surface having the appropriate surface groups, e.g., —OH groups, present on its surface, is obtained. In one aspect, the synthesis protocol is carried out under anhydrous conditions and reactions are carried out in a non-aqueous, typically organic solvent layer on the substrate surface. Suitable solvents include, but are not limited to, acetonitrile, adiponitrile, propylene carbonate, and the like.

First residues of each nucleic acid to be synthesized are covalently attached to the substrate surface via reaction with the surface bound attachment moiety (e.g., —OH groups). Depending on whether the first nucleotide residue of each nucleic acid to be synthesized on the array is the same or different, different protocols for this step may be followed. Where each of the nucleic acids to be synthesized on the substrate surface have the same initial nucleotide at the 3′ end, the entire surface of the substrate is contacted with the blocked, activated nucleoside under conditions sufficient for coupling of the activated nucleoside to the reactive groups, e.g., —OH groups, present on the substrate surface to occur. The entire surface of the array may be contacted with the fluid composition containing the activated nucleoside using any convenient protocol, such as flooding the surface of the substrate with the activated nucleoside solution, immersing the substrate in the solution of activated nucleoside, etc. The fluid composition typically includes a fluid composition of the blocked nucleoside in an organic solvent, e.g., acetonitrile, where the fluid composition may include an activating agent, e.g., tetrazole, benzoimidazolium triflate (“BZT”), S-ethyl tetrazole, and dicyanoimidazole, etc.

Alternatively, in one aspect, where the initial residue of the various nucleic acids differs among the nucleic acids, one or more sites on the substrate surface are individually contacted with a fluid composition of the appropriate blocked, activated nucleoside. Of particular interest in many embodiments is the use of pulse-jet deposition protocols, such as those described in U.S. Pat. Nos. 6,171,797; 6,180,351; 6,232,072; 6,242,266; 6,300,137; and 6,323,043; as well as U.S. patent application Ser. No. 09/302,898 filed Apr. 30, 1999. In one aspect, two or more different fluid compositions of activated, blocked nucleosides, which fluid compositions differ from each other in terms of the activated nucleoside present therein, are each pulse-jetted onto one or more distinct locations of the surface, where the type of fluid composition pulse-jetted at the locations is dictated by the sequence of the desired nucleic acid at each location.

In another aspect, the activated nucleoside monomers employed in this attachment step of each cycle of the subject synthesis methods are blocked at their 5′-OH functionalities (ends) with an acid labile blocking group. By acid labile blocking group is meant that the group is cleaved in the presence of an acid to yield a 5′-OH functionality. The acid labile blocking group may be DMT in certain embodiments.

The above step of the subject protocols results in a “blocked reactive moiety attached substrate” where the surface of the substrate includes blocked reactive moieties, e.g., DMT-blocked nucleoside monomers, covalently attached to the surface, either directly, if the blocked reactive moieties are the first residues to be synthesized surface-bound nucleic acids, or indirectly, i.e., where blocked monomers are at the end of growing nucleic acid chains, in which case it may interchangeably be referred to as a polymer-attached substrate, where blocked monomer attached substrate is used herein for convenience.

This resultant “blocked reactive moiety attached substrate” is then subjected to the next step of the subject synthesis cycle, i.e., the generation of functional groups on the substrate surface, e.g., for use in the next round of array synthesis. In this next step, viewed as the functional group generation step, the substrate surface is sequentially contacted with at least an oxidizing agent and a deblocking agent, where representative embodiments may further include contacting the surface with a capping agent. In performing the above-described substeps, while the order of oxidation and deblocking may be reversed, the deblocking step is typically performed following capping/oxidation. As such, the capping/oxidation steps are described together first, followed by a description of the deblocking step. It should be noted that capping before oxidation also prevents formation of branched DNA, while capping after oxidation also removes moisture introduced by the oxidation. In some protocols, capping is done before and after oxidation. As such, capping may be performed before oxidation, after oxidation, or both before and after oxidation.

Representative deblocking, oxidation, capping fluids are now described. It should be noted that the following descriptions of deblocking, oxidizing, capping fluids are merely representative, and that other types of fluids may be employed in a given protocol, e.g., a combined oxidizing/deblocking fluid, such as that described in Published United States Application No. 20020058802, the disclosure of which is herein incorporated by reference.

Oxidation

Oxidation results in the conversion of phosphite triesters present on the substrate surface following coupling to phosphotriesters. Oxidation is accomplished by contacting the surface with an oxidizing solution, as described above, which solution includes a suitable oxidating agent. Various oxidizing agents may be employed, where representative oxidizing agents include, but are not limited to: organic peroxides, oxaziridines, iodine, sulfur etc. The oxidizing agent is typically present in a fluid solvent, where the fluid solvent may include one or more cosolvents, where the solvent components may be organic solvents, aqueous solvents, ionic liquids, etc. A representative oxidizing agent of interest is I₂/H₂O/Pyridine/THF. Following contact of the surface with the oxidizing solution, excess is removed as described above.

Optional Capping

In addition, unreacted hydroxyl groups may be (though not necessarily) capped, e.g., using any convenient capping agent, as is known in the art. This optional capping is accomplished by contacting the surface with an capping solution, as described above, which solution includes a suitable capping agent, such as a solution of acetic anhydride, pyridine or 2,6-lutidine (2,6-dimethylpyridine), and tetrahydrofuran (“THF”); a solution of 1-methyl-imidazole in THF; etc. Following contact of the surface with the oxidizing solution, excess oxidizing solution is removed as described above.

Deblocking

The next substep in the subject methods is the deblocking step, where acid labile protecting groups present at the 5′ ends of the growing nucleic acid molecules on the substrate are removed to provide free 5′ OH moieties, e.g., for attachment of subsequent monomers, etc. In this deblocking step (which may also be referred to as a deprotecting step as results in removal of the protecting blocking groups), the entire substrate surface is contacted with a deblocking or deprotecting agent, typically in a flow cell, as described above. The substrate surface is incubated for a sufficient period of time under appropriate conditions for all available protecting groups to be cleaved from the nucleotides that they are protecting.

In certain exemplary embodiments, the deblocking solution includes an acid present in an organic solvent, e.g., one that has a low vapor pressure. The vapor pressure of the organic solvent that is employed in the deblocking solution may be at least substantially the same as toluene, by which is meant that the vapor pressure may not be more than about 350%, e.g., may not more than about 150% of the vapor pressure of toluene at a given set of temperature/pressure conditions. In certain embodiments, the organic solvent may be one that has a vapor pressure that is less than about 13 KPa, e.g., less than about 8 KPa, e.g., less than about 5 KPa at standard temperature and pressure conditions i.e., STP conditions (0° C.; 1 ATM). Organic solvents that may be used include, but are not limited to, toluene, xylene (o, m, p), ethylbenzene, perfluoro-n-heptane, perfluoro decalin, chlorobenzene, 1,2 dichloroethane, 1,1,2 trichloroethane, 1,1,2,2 tetrachloroethane, pentachloroethane, and the like; where in certain embodiments, the organic solvent that is employed is toluene. The acid deblocking agent employed in the deblocking solution may vary, where representative acids include, but are not limited to: acetic acids, e.g., acetic acid, mono acetic acid, dichloroacetic acid, trichloroacetic acid, monofluoroacetic acid, difluoroacetic acid, trifluoroacetic acid, and the like. The amount of acid in the solution is sufficient to remove blocking groups, and may range from between about 0.1 and about 20%, e.g., from between about 1 and about 3%, as is known in the art.

Contact of the substrate surface with a deblocking agent results in removal of the protecting groups from the blocked substrate bound residues. As such, this step results in the deprotection of, for example, a nucleotide residue on the substrate surface. Following deprotection, the deblocking solution is removed from the surface of the substrate.

Removal of the deblocking agent according to the subject methods results in a substrate surface in which the surface bound moieties are deprotected. In others words, removal of the deblocking agent results in the production of an array of moieties, such as nucleotide residues, stably associated with the substrate surface, where the nucleotide residues on the array surface have 5′-OH groups available for reaction with an activated nucleotide in subsequent cycles.

As reviewed above, a feature of the subject methods is that the substrate surface is washed in a dedicated wash fluid reaction chamber, where this wash step occurs at least between the functional group generation step and blocked monomer attachment step. Furthermore, the surface of the substrate may be washed in a dedicated wash fluid reaction chamber between one or more of the above described capping, oxidation and deblocking steps, and after the deblocking step.

Any convenient wash fluid may be employed in these one or more wash steps. In certain embodiments, the wash fluid is water, an organic solvent, an ionic liquid or a mixture containing more than one type of the previous fluids. In certain embodiments, solvents of from 1 to about 6, more usually from 1 to about 4, carbon atoms, including alcohols such as methanol, ethanol, propanol, etc., ethers such as tetrahydrofuran, ethyl ether, propyl ether, dioxane, etc., acetonitrile, dimethyl-formamide, dimethylsulfoxide, and the like, may be employed. Specific organic solvents of interest include, but are not limited to: acetonitrile, acetone, methanol, ethanol and the like as well as mixtures of the like.

In the monomer attachment step of each cycle of the above described representative embodiment, one or more different reactive moieties, such as 5′OH blocked nucleoside monomers, is contacted with one or more different locations of a substrate surface that displays surface attachment moieties, such as hydroxyl functional groups, such that the reactive moieties covalently bond to the surface attachment moieties. For example, in some embodiments where the reactive moieties are 5′OH blocked nucleoside monomers, the nucleoside monomers become covalently bound to the surface, e.g., via a nucleophilic substitution reaction between the an activated (e.g., protonated) phosphoramidite moiety of the blocked nucleoside monomer and the surface displayed hydroxyl functionality.

The surface-displayed attachment moieties may be on the surface of a nascent substrate, i.e., a substrate surface that not yet include deposited monomers, or may be at the end of a growing polymeric ligand, for example, the 5′ end of a growing nucleic acid, or may be at the 3′ end of a growing nucleic acid, depending on the particular point in the synthesis protocol. For example, at the beginning of a particular synthesis protocol, the surface-displayed attachment moieties are positioned immediately on the surface of a solid support or substrate. In contrast, following one or more cycles of a given synthesis protocol, the surface displayed attachment moieties are present at the end of a growing polymeric ligand, for example, at the 5′ ends of growing nucleic acids which, in turn, are covalently bonded to the surface of the substrate.

The substrate may be any convenient substrate that finds use in biopolymeric arrays. In general, the substrate may be rigid or flexible. The substrate may be fabricated from a variety of materials. In certain embodiments, the materials from which the substrate may be fabricated may exhibit a low level of non-specific binding during hybridization events. In many situations, it is of interest to employ a material that is transparent to visible and/or UV light. Specific materials of interest include: silicon; glass; plastics, e.g., polytetrafluoroethylene, polypropylene, polystyrene, polycarbonate, and blends thereof, and the like; metals, e.g. gold, platinum, and the like; etc. The surface may include one or more different layers of compounds that serve to modify the properties of the surface in a desirable manner. Such modification layers, when present, may range in thickness from a monomolecular thickness to about 1 mm, e.g., from a monomolecular thickness to about 0.1 mm or from a monomolecular thickness to about 0.001 mm. Modification layers of interest include: inorganic and organic layers such as metals, metal oxides, conformal silica or glass coatings, polymers, small organic molecules and the like. Polymeric layers of interest include layers of: peptides, proteins, nucleic acids or mimetics thereof, e.g. peptide nucleic acids and the like; polysaccharides, phospholipids, polyurethanes, polyesters, polycarbonates, polyureas, polyamides, polyethyleneamines, polyarylene sulfides, polysiloxanes, polyimides, polyacetates, and the like, where the polymers may be hetero- or homopolymeric, and may or may not have separate functional moieties attached thereto, e.g. conjugated. The particular surface chemistry will be dictated by the specific process to be used in polymer synthesis, as described in greater detail infra. However, as mentioned above, in one aspect, the substrate that is initially employed has a surface that displays hydroxyl functional groups.

As indicated above, the above description is merely representative. Various modifications may be made and still fall within the scope of the invention. For example, other functional groups may be employed, e.g., amine functional groups. In yet other embodiments, base labile blocking groups may be employed, where such groups and the use thereof are described in U.S. Pat. No. 6,222,030. In these latter types of embodiments, the acid deblocking agent described above may be replaced with a base deblocking agent. In yet other embodiments, the “direction” of synthesis may be reversed, such that the synthesized nucleic acids are attached to the substrate at their 5′ ends and one generates 3′ functional groups in the deblocking/deprotecting step.

The subject invention has been further described above in terms of representative embodiments of fabrication of nucleic acids arrays. While the above description has been provided in terms of nucleic acid array production protocols for ease and clarity of description, the scope of the invention is not so limited, but instead extends to the fabrication of any type of array structure, particularly biopolymeric array structure, including, but not limited to polypeptide arrays, in addition to the above described nucleic acid arrays. The synthesis of polypeptides involves the sequential addition of amino acids to a growing peptide chain. This approach includes attaching an amino acid to the functionalized surface of the support. In one approach the synthesis involves sequential addition of carboxyl-protected amino acids to a growing peptide chain with each additional amino acid in the sequence similarly protected and coupled to the terminal amino acid of the oligopeptide under conditions suitable for forming an amide linkage. Such conditions are well known to the skilled artisan. See, for example, Merrifield, B. (1986); Solid Phase Synthesis, Sciences 232, 341-347. After polypeptide synthesis is complete, acid is used to remove the remaining terminal protecting groups. In accordance with embodiments of the present invention each of certain repetitive steps involved in the addition of an amino acid may be carried out in a flow cell. Such repetitive steps may involve, among others, washing of the surface, protection and deprotection of certain functionalities on the surface, oxidation or reduction of functionalities on the surface, and so forth. The subject invention is particularly useful for the fabrication of arrays using a protocol that includes a deblocking step, such as the representative deblocking step described above, where a blocking group is removed at some point during an iterative synthesis process.

The subject invention also provides apparatuses for producing a chemical array where embodiments include: (a) a fluid deposition element for depositing a fluid droplet on a substrate surface; (b) a reagent contact reaction chamber for contacting the substrate surface with one or more reagents, such as an oxidizing agent and a deblocking agent; and (c) a dedicated wash fluid reaction chamber different from the reagent contact reaction chamber. In certain embodiments the reaction chambers are flow cells.

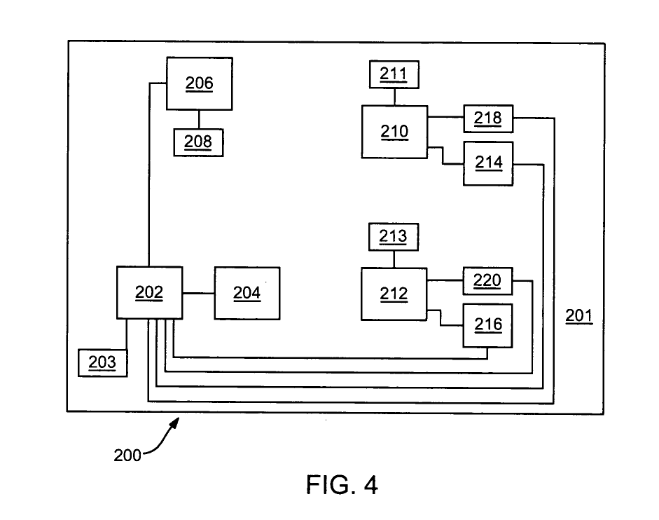

One representative embodiment of an apparatus in accordance with the present invention is depicted in FIG. 4 in schematic form. Apparatus 200 includes platform 201 on which the components of the apparatus are mounted. Apparatus 200 includes main computer 202, with which various components of the apparatus are in communication. Video display 203 is in communication with computer 202. Apparatus 200 further includes fluid deposition element 204, which is controlled by main computer 202. The nature of fluid deposition element 204 depends on the nature of the deposition technique employed to add fluid to the substrate surface. Such deposition techniques include, by way of illustration and not limitation, printing techniques, such as pulse-jet deposition printing, and so forth. Transfer robot 206 is also controlled by main computer 202 and includes a robot arm 208 that moves a substrate from fluid deposition element 204 to first reaction chamber 210 then to second reaction chamber 212 (or to any other position such as to and/or from a printing chamber). In one embodiment robot arm 208 introduces a substrate fluid deposition element 204 horizontally for depositing a fluid droplet on the substrate surface and then introduces the substrate into first reaction chamber 210 for contacting the substrate surface with one or more agents, such as an oxidizing agent and a deblocking agent, then introduces the substrate into second reaction chamber 212 for contacting the substrate surface with a washing agent. Mechanisms for rotating a substrate are described herein and include, but are not limited to, pneumatic pistons, belt or chain drives, cams and followers, rack and pinions or other gear drives, lead screws, direct drive motors, etc, which may be controlled by a processor.

First reaction chamber 210 is in communication with program logic controller 214 (which corresponds to a controller (not shown), which is controlled by main computer 202, and second reaction chamber 212 is in communication with program logic controller 216, which is also controlled by main computer 202. First reaction chamber 210 assembly is in communication with fluid dispensing station 211 and flow sensor and level indicator 218, which are controlled by main computer 202, and second reaction chamber 212 is in communication with fluid dispensing station 213 and flow sensor and level indicator 220, which are also controlled by main computer 202.

In some embodiments, the apparatus of the invention may optionally include at least one additional reaction chamber. As such, the apparatus of the invention may also include one or more different reaction chambers for contacting the substrate surface with an agent different than the agent of the first and second reaction chambers, such as a capping agent and a washing agent. For example, the subject apparatus may include a third reaction chamber for contacting the substrate surface with a capping agent after contacting the substrate surface with an oxidizing agent, and a fourth reaction chamber for contacting the substrate surface with a washing agent between one or more of the described oxidation, capping, and deblocking steps.

The apparatus of the invention further includes appropriate electrical and mechanical architecture and electrical connections, wiring and devices such as timers, clocks, and so forth for operating the various elements of the apparatus. Such architecture is familiar to those skilled in the art and will not be discussed in more detail herein.

The methods in accordance with the present invention may be carried out under computer control, that is, with the aid of a computer. For example, an IBM® compatible personal computer (PC) may be utilized. The computer may be driven by software specific to the methods described herein. Computer hardware capable of assisting in the operation of the methods in accordance with the present invention involves in certain embodiments a system with at least the following specifications: Pentium® processor or better with a clock speed of at least 100 MHz, at least 32 megabytes of random access memory (RAM) and at least 80 megabytes of virtual memory, running under either the Windows 95 or Windows NT 4.0 operating system (or successor thereof). Software that may be used to carry out the methods may be, for example, Microsoft Excel or Microsoft Access, suitably extended via user-written functions and templates, and linked when necessary to stand-alone programs. Examples of software or computer programs used in assisting in conducting the present methods may be written, preferably, in Visual BASIC, FORTRAN and C++. It should be understood that the above computer information and the software used herein are by way of example and not limitation. The present methods may be adapted to other computers and software. Other languages that may be used include, for example, PASCAL, PERL or assembly language.

A computer program may be utilized to carry out the above method steps. The computer program provides for controlling the valves of the flow assemblies to introduce reagents into the flow cells, vent the flow cells, and so forth. The computer program further may provide for moving the substrate to and from a station for monomer addition at a predetermined point in the aforementioned method.

Another aspect of the present invention is a computer program product including a computer readable storage medium having a computer program stored thereon which, when loaded into a computer, performs the aforementioned method.

In exemplary embodiments, the methods are coded onto a computer-readable medium in the form of programming.

The data storage means may include any manufacture including a recording of the present information as described above, or a memory access means that can access such a manufacture.

In certain embodiments, a processor of the subject invention may be in operable linkage, i.e., part of or networked to, the aforementioned device, and capable of directing its activities.

A processor may be pre-programmed, e.g., provided to a user already programmed for performing certain functions, or may be programmed by a user, where a processor may be programmed, e.g., by a user, from a remote location meaning a location other than the location at which the processor and/or flow cell and/or substrate is present. For example, a remote location could be another location (e.g. office, lab, etc.) in the same city, another location in a different city, another location in a different state, another location in a different country, etc. A processor may be remotely programmed by “communicating” programming information to the processor, i.e., transmitting the data representing that information as fix this—electrical signals over a suitable communication channel (for example, a private or public network). “Forwarding” programming refers to any means of getting that programming from one location to the next, whether by physically transporting that programming or otherwise (where that is possible) and includes, physically transporting a medium carrying the programming or communicating the programming. Any convenient telecommunications means may be employed for transmitting the programming, e.g., facsimile, modem, Internet, LAN, WAN or other network means, etc.

Also provided by the subject invention are chemical arrays, such as nucleic acid arrays, produced according to the subject methods, as described above. Exemplary nucleic acid arrays include at least two distinct nucleic acids that differ by monomeric sequence immobilized on, e.g., covalently to, different and known locations on the substrate surface. In certain embodiments, each distinct nucleic acid sequence of the array is typically present as a composition of multiple copies of the polymer on the substrate surface, e.g., as a spot on the surface of the substrate. The number of distinct nucleic acid sequences, and hence spots or similar structures, present on the array may vary, but is generally at least 2, usually at least 5 and more usually at least 10, where the number of different spots on the array may be as a high as 50, 100, 500, 1000, 10,000 or higher, depending on the intended use of the array. The spots of distinct polymers present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm2, where the density may be as high as 106 or higher, but will generally not exceed about 105 spots/cm2. In other embodiments, the polymeric sequences are not arranged in the form of distinct spots, but may be positioned on the surface such that there is substantially no space separating one polymer sequence/feature from another.

As indicated above, the chemical arrays may be arrays of nucleic acids, including oligonucleotides, polynucleotides, DNAs, RNAs, synthetic mimetics thereof, and the like.

A feature of the subject arrays, which feature results from the protocol employed to manufacture the arrays, is that each probe location of the arrays is highly uniform in terms of probe composition, since the entire substrate surface is exposed to each reagent for the same period of time with the same concentration of reagents, regardless of the densities of the fluids, e.g., regardless of the densities of two sequentially contacting fluids during the functional group generation step. As such, embodiments include arrays wherein the proportion of full-length sequence within each feature is higher as compared to arrays produced using analogous protocols but not the subject to positioning of the substrate based on fluid densities during a functional group generation step, as described herein (e.g., at least about 1-fold higher, often at least about 2-fold higher, such as at least about 25-, 50- or 75-fold higher), and the length distribution within each feature is less skewed towards shorter sequences. As a result, background noise and non-selective signal may be reduced in the hybridization signal, and sensitivity and specificity improved.

The apparatus and methods of the present invention are particularly useful in the synthesis of chemical arrays, including biopolymeric arrays, such as polypeptide and nucleic acid (e.g., oligonucleotide) arrays.

Chemical arrays produced as described above find use in a variety of different applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g. an isotopic or fluorescent label present on the analyte, etc. The presence of the analyte in the sample is then deduced from the detection of binding complexes on the substrate surface.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g. a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992. Also of interest are U.S. Pat. Nos. 6,656,740; 6,613,893; 6,599,693; 6,589,739; 6,587,579; 6,420,180; 6,387,636; 6,309,875; 6,232,072; 6,221,653; and 6,180,351. In certain embodiments, the subject methods include a step of transmitting data from at least one of the detecting and deriving steps, as described above, to a remote location.

Where the arrays are arrays of polypeptide binding agents, e.g., protein arrays, specific applications of interest include analyte detection/proteomics applications, including those described in U.S. Pat. Nos. 4,591,570; 5,171,695; 5,436,170; 5,486,452; 5,532,128 and 6,197,599 as well as published PCT application Nos. WO 99/39210; WO 00/04832; WO 00/04389; WO 00/04390; WO 00/54046; WO 00/63701; WO 01/14425 and WO 01/40803—the disclosures of which are herein incorporated by reference.

As such, in using an array made by the method of the present invention, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition). The results of the reading (processed or not) may be forwarded (such as by communication) to a remote location if desired, and received there for further use (such as further processing).

In certain embodiments, the methods include a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as signals (e.g., electrical, optical, radio signals, and the like) over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in using an array made by the method of the present invention, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER scanner available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

Kits for use in analyte detection assays are also provided. The kits at least include the arrays of the invention. The kits may further include one or more additional components necessary for carrying out an analyte detection assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the array assay devices for carrying out an array based assay. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette.

The following examples are offered by way of illustration and not by way of limitation.

### EXAMPLES

DNA microarrays were manufactured on 6^{5/8}×6 inch wafers on an automated tool designed by Agilent Technologies, Inc. using the standard phosphoramidite chemistry with the following major modifications. First, the solid support used was a flat, non-porous surface rather than a curveted, porous surface. Second, the coupling step was controlled in space using inkjet-printing technologies to deliver the appropriate amount of activated phosphoramidite to the appropriate spatial location on the solid support. Third, the oxidation and deblock reaction were performed in dedicated flowcells with approximate volumes of 20 mL each. The DNA sequences synthesized on each microarrays were proprietary sequences used to assay the quality of the synthesis and the microarrays were therefore hybridized with the appropriate, fluorescently labeled, complementary sequences. Scanning was performed on a standard Agilent scanner and data analysis was performed according to standard internal quality control methods.

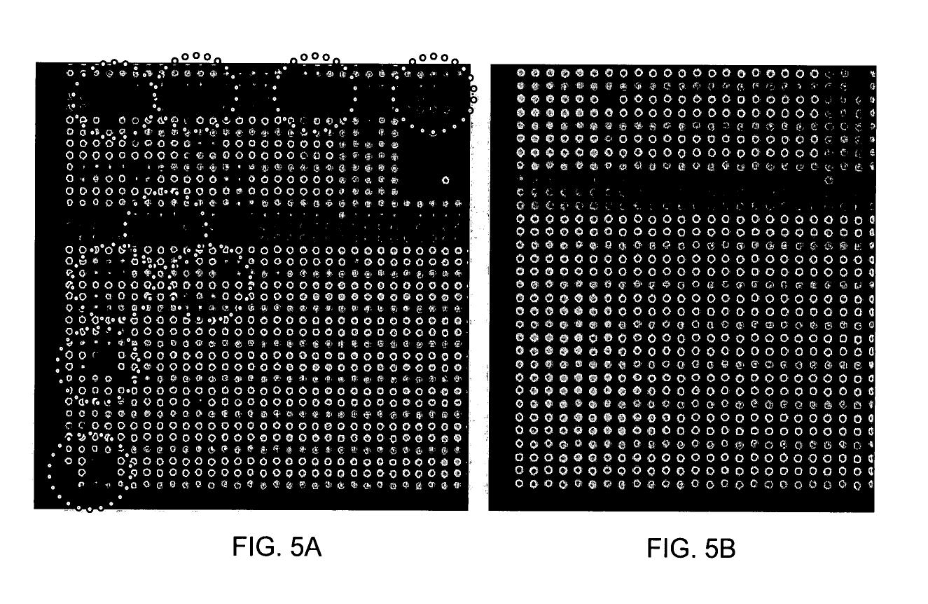

First, a wafer was synthesized without a wash step in a dedicated wash flowcell and the results are shown on FIG. 5A. As is illustrated in FIG. 5A, with dotted circles, a number of areas had individual features that had an unexpected signal levels indicating a failure in the synthesis cycle. Further experiments determined that these failures were due to the presence on the solid support of some of the active reagents used in the oxidation and deblock steps prior to coupling of droplets. Those reagents interfered with the phosphoramidite chemistry and resulted in incomplete coupling, and hence inappropriate synthesis quality as observed in the Quality Control assays.

To remediate the failure documented above, a dedicated wash step was introduced following deblock and prior to the spatially controlled coupling step. This dedicated wash step consisted in 1) moving the solid support in a dedicated flowcell not connected to the active reagents used in the oxidation and deblock manufacturing process, 2) contacting the solid support with Acetonitrile for 20 sec, 3) drying the solid support with the appropriate amount of N₂ and 4) moving the solid support out of the dedicated wash flowcell to perform the next step of the manufacturing process (spatially controlled coupling). All other steps of the manufacturing and analysis process were kept the same. As can be seen on FIG. 5B, this modified protocol resulted in no failed sequences indicating that the coupling failures were eliminated.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of producing a chemical array, said method comprising:
(a) contacting a surface of a substrate with at least a deblocking reagent in a first reaction chamber to produce substrate having a deblocked surface;
(b) washing said substrate having a deblocked surface in a second dedicated wash fluid reaction chamber to produce a substrate having a washed deblocked surface; and
(c) contacting said washed deblocked surface of said substrate with reactive moieties to covalently bond said reactive moieties to functional groups displayed on said washed deblocked surface in a manner effective to produce a chemical array.

2. The method according claim 1, further comprising reiterating steps (a), (b) and (c) at least once to produce a chemical array.

3. The method according claim 1, wherein said method further comprises contacting said surface with an oxidizing agent prior to said deblocking agent.

4. The method according to claim 3, wherein said oxidizing agent and said deblocking agent are contacted with said surface in the same reaction chamber.

5. The method according to claim 3, wherein said oxidizing agent and said deblocking agent are contacted with said surface in different reaction chambers.

6. The method according to claim 1, wherein said first and second reaction chambers are flow cells.

7. The method according to claim 1, wherein said reactive moieties are blocked reactive moieties.

8. The method according to claim 7, wherein said blocked reactive moieties are blocked nucleoside monomers.

9. The method according to claim 1, wherein said reactive moieties are contacted with said surface in a spatially controlled manner.

10. The method according to claim 9, wherein said spatially controlled manner comprises pulse-jet deposition.

11. The method according to claim 1, wherein said chemical array comprises at least two different polymeric ligands.

12. The method according to claim 11, wherein said polymeric ligands are nucleic acids.

13. The method according to claim 12, wherein said polymeric ligands are peptides.

14. A method of producing a nucleic acid array, said method comprising:
(a) producing surface attachment moieties on a surface of a substrate by
(i) contacting said surface with an oxidizing agent to produce an oxidized surface; and
(i) contacting said oxidized surface with a deblocking agent to produce a deblocked surface;
(b) washing said substrate having a deblocked surface in a dedicated wash fluid flow cell to produce a substrate having a washed deblocked surface; and
(c) contacting said washed deblocked surface with at least two different blocked nucleoside monomers in a spatially controlled manner to covalently bond said blocked nucleoside monomers to functional groups displayed on said washed deblocked surface to produce a nucleic acid array.

15. The method according claim 14, further comprising reiterating steps (a), (b) and (c) at least once.

16. The method according to claim 14, wherein said oxidizing agent and said deblocking agent are contacted with said surface in the same reagent contact flow cell that is distinct from said dedicated wash fluid flow cell.

17. The method according to claim 14, wherein said oxidizing agent and said deblocking agent are contacted with said surface in different reagent contact flow cells that are distinct from said dedicated wash fluid flow cell.

18. The method according to claim 14, wherein said spatially controlled manner comprises pulse-jet deposition.

19. An apparatus for producing a chemical array, said apparatus comprising:
(a) a spatially controlled fluid deposition element;
(b) a dedicated wash fluid reaction chamber; and
(c) a reagent contact reaction chamber.

20. The apparatus according to claim 19, further comprising at least two different reagent contact reaction chambers.

21. The apparatus according to claim 19, wherein said spatially controlled fluid deposition element is a pulse-jet.

22. The apparatus according to claim 19, wherein said reaction chambers are flow cells.

23. The apparatus according to claim 19, further comprising a mechanism for transporting a substrate between said fluid deposition element said reaction chambers.

24. A method of producing a chemical array, said method comprising:
(a) deblocking a substrate surface to produce substrate having a deblocked surface; and
(b) contacting said deblocked surface with reactive moieties to covalently bond said reactive moieties to functional groups displayed on deblocked surface in a manner effective to produce a chemical array;
wherein said method further comprises washing said substrate in a dedicated wash fluid reaction chamber.

25. The method according to claim 24, wherein said washing step occurs between steps (a) and (b).

26. The method according to claim 25, further comprising reiterating steps (a), (b) and said washing step at least once to produce a chemical array.

27. The method according to claim 24, wherein said dedicated wash fluid reaction chamber is a flow cell.

28. The method according to claim 24, wherein said deblocking comprises contacting said substrate surface with a deblocking agent in a non-spatially controlled manner such that the entire surface of said substrate is deblocked.

29. The method according to claim 24, wherein said deblocking comprises irradiating said surface in a spatially controlled manner such that said substrate surface is selectively deblocked.
